(19) United States
(12) Patent Application Publication
(10) Pub. No.: US 2002/0071861 A1
(43) Pub. Date: Jun. 13, 2002

(54) System for dermal or transdermal application containing an iota-carrageenan and process for its preparation

(76) Inventors: Mikaelle Blanchin, Seine (FR); Laurent Artaud, Joinville Le Pont (FR)

Correspondence Address: Stephen B. Maebius, FOLEY & LARDNER, Sutie 500, 3000 K. Street, N.W., Washington, DC, 20007-5109, US

(21) Appl. No.: 09/860,757

(22) Filed: May 21, 2001

Related U.S. Application Data
(60) Provisional application No. 60/205,563, filed on May 22, 2000.

(51) Int. Cl. A61K 9/70; A61K 9/14; A61K 31/715; A61K 31/717

(52) U.S. Cl. 424/449; 424/488; 514/54; 514/57

(57) ABSTRACT

The present invention provides a system for dermal or transdermal application containing a matrix based on an aqueous-alcoholic gel which contains at least one iota-type carrageenan having a molecular weight of between 300,000 D and 800,000 D, said carrageenan having a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s), and such that an aqueous solution of this carrageenan has a thixotropic gel structure.

The present invention relates to a hydrogel formulation containing at least one polysaccharide which is an iota-type carrageenan as gelling agent for the production of systems for dermal or transdermal application in particular for the release of moisturizing, dermo tightening, immunoprotective, antirubescent and soothing cosmetic ingredients, or alternatively ingredients known for their activity in dermatology.

The objectives sought by the production of systems for dermal or transdermal application are the mechanical properties of the gel on the one hand, and the adhesion of said system to the skin on the other hand. The mechanical properties of the gel must be satisfactory in order to obtain adequate elasticity and strength, with stability of these properties with respect to temperature and an absence of syneresis. The adhesiveness for its part must be adequate in order to be able to keep the system in place for the expected duration of the local treatment.

Most prior art hydrogels are coated on woven or unwoven supports; the systems of the plaster or patch type thus prepared allow use in a unit form intended to be applied to the desired part of the skin.

Some of these formulations make it possible, however, to proceed via casting and to obtain a unit form requiring no support, that is to say requiring no woven or unwoven backing. The process for manufacturing these films generally consists in preparing a mixture of gelling agents in an aqueous-alcoholic solution and then heating this mixture to a high temperature in order to work at a viscosity allowing coating or casting in a thermoformed mold, the gel then solidifies after cooling.

The principal advantage of these systems for dermal or transdermal application is that they allow their users to benefit from the skin cooling effect offered by hydrogels (because of the high proportions of water which are contained in these gels), and to provide consumers with a form which is convenient to use, making it possible to target an action on a precise location on the face or any other part of the body.

In contrast to conventional cosmetic forms such as creams or gels for spreading, the targeting makes it possible to avoid the spreading of a formula containing certain potentially irritant ingredients on part of the skin which does not require any treatment. Moreover, the spreading, by the consumer, of a conventional formula does not make it possible to precisely control the dose of active ingredient deposited on the skin, which may result in a lack of efficacy of the cosmetic product if the dose is too small, or, conversely, in unnecessary wasting of product in excess, an aspect which may be important when the formulas incorporate expensive ingredients.

Whether plasters known for their medical use or hydrogels in the form of films used for cosmetic applications are involved, none of the products currently available is completely satisfactory for the reasons which can be summarized as follows

The presence of gelatin in these products makes it possible to obtain the desired mechanical properties, but the use of gelatin in cosmetics is excluded (because of its animal origin) in a large number of countries taking interest in this market.

In the absence of gelatin, the mechanical characteristics of the films described are not satisfactory for a cosmetic use where the elasticity is important for application to the desired areas of the face. The strength of the film desired should also allow adequate retention on an unwoven support and allow easy removal, without leaving gel residues on the skin.

The gelling agent used in the manufacture of the gel should itself exhibit a moisturizing power without being too occlusive.

Finally, a very important criterion for achieving the defined objectives for a cosmetic application lies in the process used for the preparation of these hydrogel films. The processes used for the manufacture of known systems require relatively high temperatures (above 80° C.) to allow use of the gel by coating or molding. These temperatures are incompatible with the stability of cosmetic active ingredients which are potentially valuable for this application. It would be preferable to work at temperatures below 75° C. or even 65° C. or to provide a process which makes it possible to work at high temperatures but for very short durations.

In conclusion, the limits of the various systems available can be summarized as follows: inadequate gel strength for application to the face, thermal reversibility, syneresis, excessively high manufacturing temperature, presence of gelatin, presence of residues on removal of the system.

The aim of the present invention has been the development of a system for dermal or transdermal application containing no gelatin and whose properties of gel strength, elasticity, sticking power and retention on a support are comparable to those of gels based on gelatin while exhibiting a relatively low gelling temperature.

Patent Application EP 161 681 filed on May 15, 1985 describes the manufacture of a gel containing a polysaccharide and an aqueous solution of a polyalcohol. The aim sought by this application is the production of a gel which is sufficiently strong so that the use of a support can be omitted.

It is evident from the examples that the properties (gel strength, elasticity and bonding strength) of these gels are very disparate; consequently, it is not possible to deduce from EP 161 681 the preparation of a gel which can be used as a system for dermal or transdermal application according to the present invention.

In the context of the present invention, it has been demonstrated that the use of at least one iota-type carrageenan having a molecular weight of between 300,000 D and 800,000 D makes it possible to prepare a system for dermal or transdermal application which exhibits properties comparable to those of gels based on gelatin. Said carrageenan possesses a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s); an aqueous solution of this carrageenan has, in addition, a thixotropic gel structure.

Carrageenans are polysaccharides of plant origin; they are extracted in the hot state from red algae of the Rhodophyceae family (Chondrus crispus and Eucheuma spinosum).

They are copolymers of sulfate esters of D-galactose and of 3,6-anhydro-D-galactose; they may be provided in the form of potassium or calcium salts. These macromolecules are capable of binding a large quantity of water.

Their gelling occurs during cooling in a precise and relatively high temperature region which depends on the concentration and the type of carrageenan as well as the salts present.

Carrageenan gels are thermoreversible. Repeated temperature cycles (hot/cold) cause, at neutral pH, only a slight modification of the gel structure.

There are three types of carrageenans which are distinguishable by their solubility and their mechanical properties. The solubility is directly linked to the number of negative charges of the sulfate groups.

The kappa-carrageenans (1 sulfate group per 2 galactoses) : they have a high gelling power with K⁺ ions; the three-dimensional network formed is very rigid. They are only soluble in the hot state (80° C.).

The gel formed is hard, opaque and substantial syneresis is observed.

The iota-carrageenans (2 sulfate groups per 2 galactoses): they are gel-forming; a three-dimensional network is formed by joining regions with Ca⁺⁺ ions between the macromolecules. They are partially soluble in the cold state.

The gel formed is elastic, cohesive and transparent.

The lambda-carrageenans (3 sulfate groups per 2 galactoses): they are nongelling and they are soluble in the cold state. They are thickening agents and they form a viscous solution through the presence of molecules used in the medium.

For each type of carrageenan, the three forms coexist, but one is predominant, which explains the specific mechanical characteristics of the different grades of the same family.

The carrageenans used as an essential means for the present invention are iota-type carrageenans having a molecular weight of between 300,000 and 800,000 Daltons, having a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s); in addition, an aqueous solution of this carrageenan has a thixotropic gel structure.

“Thixotropic gel structure” for the purposes of the present description is understood to mean that the solution containing iota-carrageenans which is used for the preparation of the system for dermal or transdermal application should liquefy upon stirring at room temperature and recover its gelatinous consistency on standing.

In general, the iota-carrageenans used are such that they have granules which are predominantly (more than 95%) less than 250 μm.

The matrix used to form the systems for dermal or transdermal application according to the present invention is an aqueous-alcoholic matrix which comprises at least one polyalcohol in a water:polyalcohol ratio preferably of between 40:60 and 60:40. Said polyalcohol is preferably chosen from the group consisting of glycerol, sorbitol, glucose, ethylene glycol, diethylene glycol, triethylene glycol, various grades of polyethylene glycol, butylene glycol and butanediol.

The essential functions of this polyalcohol are to retain the water and, where appropriate, to facilitate the absorption of the active ingredients released at the level of the skin.

The system for dermal or transdermal application according to the present invention may also contain at least one cellulosic polymer such as for example sodium carboxymethylcelluloses or hydroxypropylmethylcellulose, polycarboxylic acids, xanthans, guar gums, pectins and alginates.

Said system for dermal or transdermal application may also comprise at least one additive chosen from the group consisting of inorganic pigments, perfumes, colorants, preservatives, solubilizing agents and pH-regulating agents.

The system for application may, depending on the use for which it is intended, comprise a support; this support may be woven or unwoven.

In addition, this system for dermal or transdermal application may be used solely for cooling purposes, but it may also contain at least one active ingredient.

All the active ingredients which are capable of being introduced into a conventional system for dermal or transdermal application may be envisaged and in particular a cosmetic active ingredient chosen from the group consisting of moisturizing agents, dermotightening agents, antistress agents, antibag agents and concealing agents may be used.

Among the preferred active ingredients, there will be noted:

the moisturizing agents: Unimoist U125 (ADF), Phytantriol (Laserson), Iricalmine (SACI CFPA),

the dermotightening agents: Pentacare HP (saci CFPA)

the antistress agents: Biomoduline (Greentech), CM Glucan (A. ARNAUD),

the antibag/concealing agents: Dextran (Soshibo), Permethol (Soshibo), Phytotal VT (Sederma), red vine extract.

The active ingredients of the field of dermatology which can be used are chosen from the group consisting of cicatrizing agents such as acexamic acid, dermocorticoids, antibacterial agents in particular fusidic acid, aminoglycosides, sulfamides, antifungal agents, antiparasitic agents, antiseptics such as quaternary ammoniums, hexamidine, copper and/or zinc salts, antiherpetic agents, antiacne agents, antiseborrheic agents, agents for cleansing and irrigating wounds and keratolytic agents such as antiverruca and anticallus agents.

The present invention therefore relates to a system for dermal or transdermal application containing a matrix based on an aqueous-alcoholic gel which contains at least one iota-type carrageenan having a molecular weight of between 300,000 D and 800,000 D, said carrageenan having a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s), and such that an aqueous solution of this carrageenan has a thixotropic gel structure.

The system for dermal or transdermal application according to the invention may further comprise at least a second iota-type carrageenan having a molecular weight of between 300,000 D and 800,000 D such that the mixture of these two carrageenans has a calcium sulfate content such that the viscosity at 20° C. of an aqueous solution containing 1% by weight of this mixture is about 120 cps (0.12 Pa.s) and that of an aqueous solution containing 2% by weight is about 1000 cps (1 Pa.s), and such that an aqueous solution of this mixture has a thixotropic gel structure.

Preferably, at least one of the iota-type carrageenans used comprises a number of D-galactose units of between 1296 and 3456. A preferred embodiment of the system for dermal or transdermal application according to the invention is such that the aqueous-alcoholic gel:polysaccharide mass ratio of said system is between 99:1 and 80:20, still more preferably between 99:1 and 90:10.

The present invention also relates to a process for using the system for dermal or transdermal application.

In addition to the advantages of the system for dermal or transdermal application according to the present invention which have already been presented, it has also been demonstrated that the composition selected made it possible to manufacture this system for dermal or transdermal application according to a simple procedure, while relatively low temperatures are maintained.

Indeed, while the temperatures generally used in this type of industry are greater than 80° C., the hydrogel composition according to the present invention makes it possible to carry out its manufacture at a temperature of less than 75° C. or even less than 65° C.

The process for preparing a system for dermal or transdermal application according to the present invention is such that an aqueous-alcoholic gel is produced by carrying out the following steps:

a) dispersing in an alcoholic phase, containing at least one polyalcohol, iota-carrageenans and optionally additives which are soluble in said alcoholic phase with stirring at room temperature,

b) heating an aqueous phase to a temperature of about 65° C., with stirring,

c) mixing the aqueous and alcoholic phases, with stirring,

and then a film is produced by coating the aqueous-alcoholic gel between thermoregulated coating means above the gelling temperature of said gel on a peelable support or by molding said gel by means of thermoformed plastic molds.

According to one variant, said process for preparing the system for dermal or transdermal application comprises the addition of an active ingredient to the aqueous-alcoholic gel in the alcoholic phase during step a), to the aqueous phase in step b), to the mixture of the aqueous and alcoholic phases in step c), or over the coated film after its passage between the thermoregulated coating means.

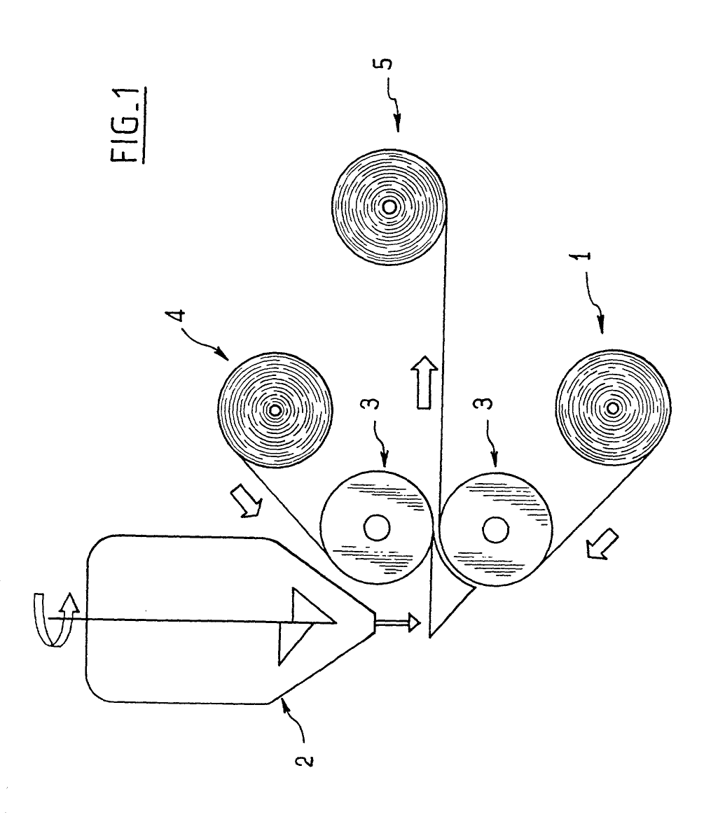

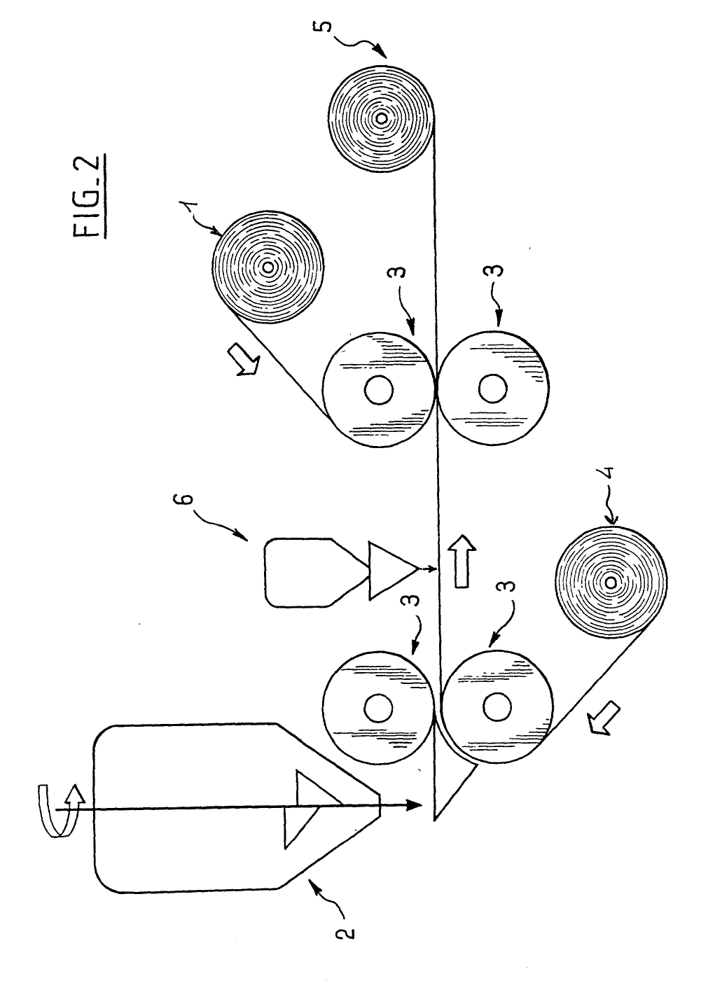

FIGS. 1 and 2 represent two embodiments of the process for preparing the system for dermal or transdermal application according to the invention; these embodiments illustrate the invention without limiting the scope thereof.

FIG. 1 is a diagram which summarizes the essential steps of an embodiment of a simple system for dermal or transdermal application. This process applies to the manufacture of a system for dermal or transdermal application comprising a woven or unwoven retaining support and which does not contain any active ingredients.

According to said FIG. 1, a roll for unwinding a peelable support (1) is unwound, bringing said peelable support to a coating zone. Said coating zone comprises a container (2) for preparing the hydrogel and the coating means. In the preparation container, the hydrogel is kept stirring at a temperature greater than its gelling temperature. The hydrogel is deposited on the peelable support at a controlled flow rate. The woven or unwoven support wound on a roll for unwinding (4) is brought to the coating zone and applied over the hydrogel layer. The “peelable support+hydrogel+woven or unwoven support” system is then brought between coating means (3) where it is flattened. The “peelable support +hydrogel+woven or unwoven support” system thus obtained is then precut into unit forms. For its packaging, the strip of system for dermal or transdermal application in unit form thus obtained is wound on a roll for winding (5).

FIG. 2 is a diagram which illustrates a process for manufacturing a system for dermal or transdermal application comprising a woven or unwoven retaining support and which contains a heat-sensitive active ingredient which is likely to be degraded if it is heated to a temperature greater than the gelling temperature of the hydrogel.

According to said FIG. 2, a roll for unwinding the woven or unwoven support (4) is unwound, bringing said peelable support to a coating zone. Said coating zone comprises a container (2) for preparing the hydrogel and a coating system. In the preparation container, the hydrogel is kept stirring at a temperature greater than its gelling temperature. The hydrogel is deposited on the woven or unwoven support at a controlled flow rate, the “woven or unwoven support+hydrogel” system is then brought between coating means (3) where it is flattened. The “woven or unwoven support+hydrogel” system is then brought to a zone for depositing the active ingredient. This zone for depositing the active ingredient comprises a container (6) containing the active ingredient and a means allowing the deposition of this active ingredient at a controlled flow rate. The peelable support wound on a roll for unwinding (1) is brought to the coating zone and applied to the hydrogel layer. The “woven or unwoven support+hydrogel+peelable support” system is then brought between other coating means (3) where it is flattened. The “woven or unwoven support+hydrogel+peelable support” system thus obtained is then precut into unit forms. For its packaging, the strip of system for dermal or transdermal application in unit form thus obtained is wound on a roll for winding (5).

The examples which follow illustrate the invention without limiting the scope thereof.

### EXAMPLES

Several systems for dermal or transdermal application of various compositions were prepared according to the same process. The properties of these systems were compared with the properties of a gel based on gelatin.

The preparation process of Examples 1 to 7 is the following.

Demineralized water is heated to 65° C., and in another vessel the carrageenan(s) and optionally the cellulosic polymer are mixed in glycerol and the powders are dispersed. The mixture obtained is slowly poured over hot water, with vigorous stirring, and the solution is heated for a few minutes, still with stirring, so that it becomes homogeneous and fluid. Next, the hot solution is poured between the two thermoregulated rollers, over the peelable film and complexed with a woven or unwoven support. The two rollers were positioned beforehand so as to obtain a gel 700 μm thick, which corresponds to a film of 755 g/m².

The heat-sensitive additives and active ingredients are solubilized in water at room temperature and they are then sprayed over the gel for a few minutes so that the solution impregnates the gel well.

The film of gel is then cooled and tested.

The compositions of Examples 1 to 7 as well as the results of the tests to establish their properties of gel strength, elasticity, stickiness, retention on a support, transparency, temperature and gelling are presented in the following table. This table contains, in addition, the results obtained with a reference agar containing, by weight, 10% of gelatin, 25% of glycerol and 65% of demineralized water.

The carrageenans used to prepare these systems for dermal or transdermal application are the following:

Viscarin GP 209 which is essentially a lambda-carrageenan composition,

Gelcarin GP 911 which is essentially a kappa-carrageenan composition,

Viscarin SD 389 which is an iota-carrageenan composition containing dextrose,

Gelcarin GP 379 contains essentially iota-carrageenans,

Seaspen PF which is a composition of iota-carrageenans of trisodium phosphate and of calcium sulfate dihydrate.

These products are marketed by the company FMC Corporation.

TABLE 1

| CONSTITUENT | Example 1 | Example 2 | Example 3 | Example 4 | Example 5 | Example 6 | Example 7 | Reference agar |
|---|---|---|---|---|---|---|---|---|
| Viscarin GP 209 | | 2 | | | | | | |
| Gelcarin GP 911 | | 4 | 3.1 | | | | | |
| Viscarin SD 389 | | | 3.1 | 2 | 1 | 3 | 2.4 | |
| Gelcarin GP 379 | 3.5 | | | 3 | 2 | 1.8 | 1.8 | |
| Seaspen PF | | | | | 3 | 1.8 | 1.8 | |
| HPMC | | | | | | 0.3 | 0.3 | |
| Glycerol | 40 | 30 | 45 | 20 | 20 | 25 | 30 | |
| Water | 56.5 | 64 | 48.8 | 75 | 74 | 68.1 | 63.7 | |
| Gel strength | ++ | + | ++++ | ++ | +++ | +++ | +++ | ++++ |
| Elasticity | ++ | − | + | +++ | + | ++ | ++++ | ++ |
| Stickiness | ++ | ++ | − | ++ | ++ | +++ | +++ | ++ |
| Retention on a | ++ | + | + | ++ | ++ | +++ | ++++ | ++++ |
| support | | | | | | | | |
| Transparency | ++ | − | − | +++ | ++ | ++ | +++ | ++++ |
| Gelling T° C. | 70 | 75 | 75 | 63 | 65 | 65 | 63 | 80 |

It is evident from this table that the compositions whose properties are most similar to those of the reference agar are the compositions which contain only iota-carrageenans.

In addition, the compositions which comprise at least two iota-carrageenans have improved properties and the presence of a cellulosic polymer:hydroxypropyl-methylcellulose, makes it possible to obtain even better results.

The preferred system for dermal or transdermal application according to the present invention is such that it contains by weight:

6% of at least three iota-type carrageenans which are preferably 2.4% of Viscarin SD 389, 1.8% of Gelcarin GP 379 and 1.8% of Seaspen PF,

0.3% of hydroxypropylmethylcellulose

30% of glycerol and 63.7% of demineralized water in the form of an aqueous-alcoholic gel.

The systems for dermal or transdermal application according to the present invention have mechanical properties which are quite comparable to those of the reference system based on gelatin.

1. A system for dermal or transdermal application containing a matrix based on an aqueous-alcoholic gel which contains at least one iota-type carrageenan having a molecular weight of between 300,000 D and 800,000 D, said carrageenan having a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s), and such that an aqueous solution of this carrageenan has a thixotropic gel structure.

2. The system for dermal or transdermal application as claimed in claim 1, which contains at least a second iota-type carrageenan having a molecular weight of between 300,000 D and 800,000 D such that the mixture of these two carrageenans has a calcium sulfate content such that the viscosity at 20° C. of an aqueous solution containing 1% by weight of this iota-carrageenan is about 120 cps (0.12 Pa.s) and that of an aqueous solution containing 2% by weight is about 1000 cps (1 Pa.s), and such that an aqueous solution of this mixture has a thixotropic gel structure.

3. The system for dermal or transdermal application as claimed in claim 1 or 2, wherein at least one iota-type carrageenan comprises a number of D-galactose units of between 1296 and 3456.

4. The system for dermal or transdermal application as claimed in one of the preceding claims, wherein the aqueous-alcoholic gel:polysaccharide mass ratio of said system is between 99:1 and 80:20.

5. The system for dermal or transdermal application as claimed in one of the preceding claims, wherein the aqueous-alcoholic gel:polysaccharide mass ratio of said system is between 99:1 and 90:10.

6. The system for dermal or transdermal application as claimed in one of the preceding claims, which contains a cellulosic polymer.

7. The system for dermal or transdermal application as claimed in one of the preceding claims, wherein the aqueous-alcoholic gel comprises water and a polyalcohol in a water:polyalcohol mass ratio of between 40:60 and 60:40.

8. The system for dermal or transdermal application as claimed in claim 7, wherein the polyalcohol is chosen from the group consisting of glycerol, sorbitol, glucose, ethylene glycol, diethylene glycol, triethylene glycol, various grades of polyethylene glycol, butylene glycol and butanediol.

9. The system for dermal or transdermal application as claimed in one of the preceding claims, which contains at least one active ingredient.

10. The system for dermal or transdermal application as claimed in claim 9, wherein the active ingredient is a cosmetic active ingredient and is chosen from the group consisting of moisturizing agents, dermo tightening agents, antistress agents, antibag agents and concealing agents.

11. The system for dermal or transdermal application as claimed in claim 9, wherein the active ingredient belongs to the field of dermatology and is chosen from the group consisting of cicatrizing agents, dermocorticoids, antibacterial agents, antifungal agents, antiparasitic agents, antiseptics, antiherpetic agents, antiacne agents, antiseborrheic agents, agents for cleansing and irrigating wounds and keratolytic agents.

12. The system for dermal or transdermal application as claimed in one of the preceding claims, which contains at least one additive chosen from the group consisting of inorganic pigments, perfumes, colorants, preservatives, solubilizing agents and pH-regulating agents.

13. The system for dermal or transdermal application as claimed in one of the preceding claims, which contains a woven or unwoven support.

14. The system for dermal or transdermal application as claimed in one of the preceding claims, which contains by weight:
6% of at least three iota-type carrageenans which are preferably 2.4% of Viscarin SD 389, 1.8% of Gelcarin GP 379 and 1.8% of Seaspen PF,
0.3% of hydroxypropylmethylcellulose
30% of glycerol and 63.7% of demineralized water in the form of an aqueous-alcoholic gel.

15. A preparation of a system for dermal or transdermal application as claimed in one of the preceding claims, wherein an aqueous-alcoholic gel is produced by carrying out the following steps:
a) dispersing in an alcoholic phase, containing at least one polyalcohol, iota-carrageenans and optionally additives which are soluble in said alcoholic phase with stirring at room temperature,
b) heating an aqueous phase to a temperature of about 65° C., with stirring,
c) mixing the aqueous and alcoholic phases, with stirring, and then a film is produced by coating the aqueous-alcoholic gel between thermoregulated coating means above the gelling temperature of said gel on a peelable support or by molding said gel by means of thermoformed plastic molds.

16. The preparation of a system for dermal or transdermal application as claimed in claim 15, wherein there is added an active ingredient to the aqueous-alcoholic gel in the alcoholic phase during step a), to the aqueous phase in step b), to the mixture of the aqueous and alcoholic phases in step c), or over the coated film after its passage between the thermoregulated coating means.
